(19) United States
(12) Patent Application Publication
(10) Pub. No.: US 2025/0025128 A1
(43) Pub. Date: Jan. 23, 2025

(54) MIXED REALITY GUIDANCE OF ULTRASOUND PROBE

(71) Applicant: Howmedica Osteonics Corp., Mahwah, NJ (US)

(72) Inventor: Jean Chaoui, Lyon (FR)

(21) Appl. No.: 18/704,830

(22) PCT Filed: Oct. 25, 2022
(86) PCT No.: PCT/US2022/047772

§ 371 Date: Apr. 25, 2024

Related U.S. Application Data
(60) Provisional application No. 63/273,008, filed on Oct. 28, 2021.

(51) Int. Cl. A61B 8/00; A61B 90/00; A61B 90/50

(52) U.S. Cl. CPC A61B 8/42; A61B 90/37; A61B 2090/365; A61B 2090/372; A61B 2090/502

(57) ABSTRACT

Techniques and systems are described for mixed reality (MR) guidance of an ultrasound probe. A system may obtain reference data depicting a bone of a patient; determine a physical location of an ultrasound probe; generate, based on first ultrasound data generated by the ultrasound probe, registration data that registers virtual locations on the bone of the patient as depicted in the reference data with corresponding physical locations on the bone of the patient; generate virtual guidance based on the reference data, the registration data, and the physical location of the ultrasound probe, wherein the virtual guidance provides guidance to a clinician regarding how the ultrasound probe is positioned relative to a target position at which the ultrasound probe is able to generate second, ultrasound data that provides information regarding a soft tissue structure of the patient; and cause a MR visualization device to output the virtual guidance to the clinician.

This application claims the benefit of U.S. Provisional Patent Application 63/273,008, filed Oct. 28, 2021, the entire content of which is incorporated by reference.

### BACKGROUND

Planning and performing a surgery, diagnosing a condition, or performing other types of medical tasks may involve acquiring information regarding the anatomy of a patient. The information regarding the anatomy of the patient may include information regarding the bones of the patient, such as the sizes, shapes, and positions of the bones of the patient. Additionally, the information regarding the anatomy of the patient may also include information regarding various soft tissue structures of the patient, such as the locations and qualities of muscles, tendons, ligaments, cartilage, retinacula, blood vessels, and so on. Acquiring high-quality information regarding both the bones of the patient and the soft tissue structures of the patient may involve different skill sets.

### SUMMARY

This disclosure describes techniques in which mixed reality (MR) guidance is used to help a clinician position an ultrasound probe to acquire information regarding the soft tissue structures involved in a surgery, such as an orthopedic surgery. As described here, a computing system may obtain reference data that depicts at least one bone of the patient. Example types of reference data may include one or more computed tomography (CT) images, magnetic resonance imaging (MRI) images, nuclear magnetic resonance (NMR) images, and so on. Additionally, the computing system may use the reference data to generate virtual guidance. The virtual guidance provides guidance to a clinician regarding how the ultrasound probe is positioned relative to a target position at which the ultrasound probe is able to generate second ultrasound data that provides information regarding a soft tissue structure of the patient. For example, the virtual guidance may instruct the clinician how to move the ultrasound probe so that the ultrasound probe is in a target position to generate ultrasound data that provides information regarding a soft tissue structure of the patient. The computing system may cause a head-mounted MR visualization device to output the virtual guidance to the clinician.

In one example, this disclosure describes a method comprising: obtaining reference data depicting a bone of a patient; determining a physical location of an ultrasound probe; generating, based on first ultrasound data generated by the ultrasound probe, registration data that registers virtual locations on the bone of the patient as depicted in the reference data with corresponding physical locations on the bone of the patient; generating virtual guidance based on the reference data, the registration data, and the physical location of the ultrasound probe, wherein the virtual guidance provides guidance to a clinician regarding how the ultrasound probe is positioned relative to a target position at which the ultrasound probe is able to generate second ultrasound data that provides information regarding a soft tissue structure of the patient; and causing a head-mounted Mixed Reality (MR) visualization device to output the virtual guidance to the clinician.

In another example, this disclosure describes a system comprising: a memory configured to store reference data depicting a bone of a patient; processing circuitry configured to: determine a physical location of an ultrasound probe; generate, based on first ultrasound data generated by the ultrasound probe, registration data that registers virtual locations on the bone of the patient as depicted in the reference data with corresponding physical locations on the bone of the patient; generate virtual guidance based on the reference data, the registration data, and the physical location of the ultrasound probe, wherein the virtual guidance provides guidance to a clinician regarding how the ultrasound probe is positioned relative to a target position at which the ultrasound probe is able to generate second ultrasound data that provides information regarding a soft tissue structure of the patient; and cause a head-mounted Mixed Reality (MR) visualization device to output the virtual guidance to the clinician.

The details of various examples of the disclosure are set forth in the accompanying drawings and the description below. Various features, objects, and advantages will be apparent from the description, drawings, and claims.

### BRIEF DESCRIPTION OF DRAWINGS

FIG. 1 is a conceptual diagram illustrating an example system in which one or more techniques of this disclosure may be performed.

FIG. 2 is a conceptual diagram illustrating an example computing system in accordance with one or more techniques of this disclosure.

FIG. 3 is a flowchart illustrating an example operation of a system in accordance with one or more techniques of this disclosure.

FIG. 4 is a flowchart illustrating an example operation of the system for generating registration data, in accordance with one or more techniques of this disclosure.

FIG. 5 is a conceptual diagram illustrating matching curves in accordance with one or more techniques of this disclosure.

FIG. 6 is a conceptual diagram illustrating example virtual guidance during an ultrasound examination of a shoulder of a patient, in accordance with one or more techniques of this disclosure.

### DETAILED DESCRIPTION

A clinician, such as a surgeon, may need to acquire information about the bones and soft tissue of a patient before, during, or after performing a medical task, such as a surgery. For example, when planning a shoulder replacement surgery, the surgeon may need to acquire information about the scapula, humerus, and rotator cuff muscles. Computed Tomography (CT) images, and 3-dimensional (3D) models generated based on CT images, provide accurate depictions of the patient's bones. However, because CT images are generated using x-rays that easily pass through most soft tissue structures, CT images are frequently unable to provide high-quality information about the patient's soft tissue structures. On the other hand, ultrasound images are able to provide high-quality information about soft tissue structures but provide less accurate information about bones than CT images.

A clinician may need specialized training to gain the ability to position an ultrasound probe to obtain high-quality ultrasound images. For instance, it may be difficult for an untrained clinician to position an ultrasound probe to gather useful information about a specific muscle or tendon. Thus, the need for a trained ultrasound technician may increase the costs and delays associated with performing a surgery. Robotic probe positioning systems have been developed to position ultrasound probes. However, access to such robotic probe positioning systems may be limited and expensive. Moreover, robotic probe positioning systems may be obtrusive and interfere with a surgeon during a surgery.

This disclosure describes techniques that may improve the process of using an ultrasound probe to gather information for a medical task. As described herein, a computing system may obtain reference data depicting a bone of a patient. The reference data may include one or more CT images (e.g., a plurality of CT images) of the bone, a 3-dimensional (3D) model of the bone, or another type of medical image depicting the bone of the patient. Furthermore, the computing system may determine a physical location of an ultrasound probe. The computing system may generate, based on first ultrasound data generated by the ultrasound probe, registration data that registers virtual locations on the bone of the patient as depicted in the reference data with corresponding physical locations on the bone of the patient. Additionally, the computing system may generate virtual guidance based on the reference data, the registration data, and the physical location of the ultrasound probe. The virtual guidance may provide guidance to a clinician regarding how the ultrasound probe is positioned relative to a target position at which the ultrasound probe is able to generate second ultrasound data that provides information regarding a soft tissue structure of the patient. For example, the virtual guidance may instruct the clinician how to move the ultrasound probe so that the ultrasound probe is at a target position to generate second ultrasound data that provides information regarding a soft tissue structure of the patient. The computing system may also cause a head-mounted Mixed Reality (MR) visualization device to output the virtual guidance to the clinician. In this way, the clinician may be able to both see the patient and see the virtual guidance. The use of virtual guidance in this way may help the clinician obtain information about the soft tissue structure.

Moreover, in some examples, the computing system may generate a virtual model (e.g., a 2-dimensional (2D) or 3D model) of the soft tissue structure based on data generated by the ultrasound probe. The MR visualization device may output the virtual model of the soft tissue structure so that the virtual model of the soft tissue structure appears to the clinician to be superimposed on the patient at an actual location of the soft tissue structure. The MR visualization device may also output virtual models of one or more bones of the patient so that the virtual bones of the patient appear to the clinician to be superimposed on the patient at actual locations of the bones of the patient. In this way, the clinician may be able to easily comprehend the locations of hidden soft tissue structures and bones of the patient. In some examples, the computing system may output the virtual model of the soft tissue structure and virtual models of the bones causing the virtual model of the soft tissue structure and the virtual models of the bones for display on a monitor. In such examples, the clinician may use these virtual models for various purposes, such as presurgical planning.

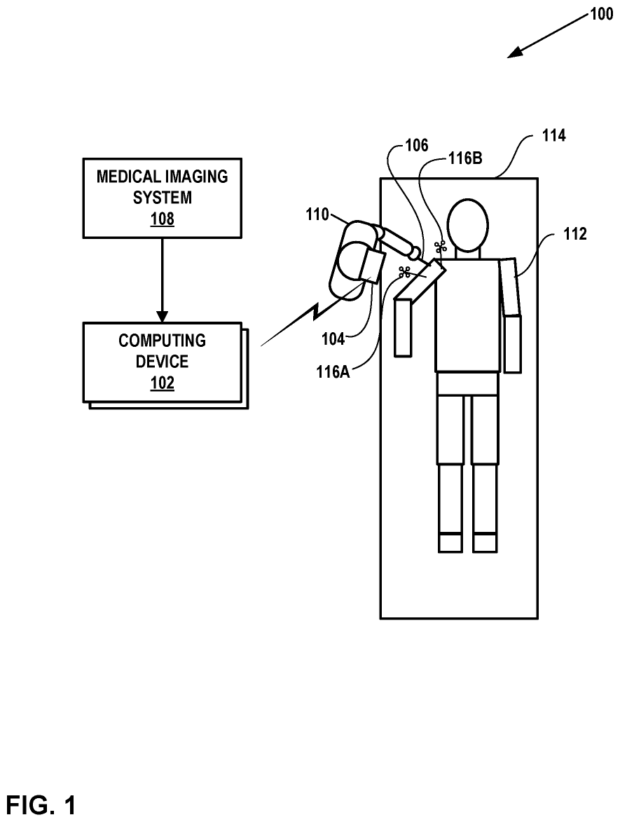

FIG. 1 is a conceptual diagram illustrating an example system 100 in which one or more techniques of this disclosure may be performed. In the example of FIG. 1, system 100 includes one or more computing devices 102, a MR visualization device 104, an ultrasound probe 106, and a medical imaging system 108. A clinician 110 is using ultrasound probe 106 to perform an examination on a patient 112 who is positioned on a table 114. Clinician 110 may be a surgeon, nurse, technician, medic, physician, or other type of medical professional or person. Clinician 110 and patient 112 do not form part of system 100. MR visualization device 104 may use markers 116A, 116B (collectively, “markers 116”) to determine a position of patient 112. Although the example of FIG. 1 shows clinician 110 performing the ultrasound examination on a shoulder of patient 112, the techniques of this disclosure may be applicable with respect to other parts of the body of patient 112, such as a foot, ankle, knee, hip, elbow, spine, wrist, hand, chest, and so on.

In general, clinician 110 performs the ultrasound examination by positioning ultrasound probe 106 on the skin of patient 112. Ultrasound probe 106 generates ultrasonic waves and detects returning ultrasonic waves. The returning ultrasonic waves may include reflections of the ultrasonic waves generated by ultrasound probe 106. Ultrasound probe 106 may generate data based on the detected returning ultrasonic waves. The data generated by ultrasound probe 106 may be processed to generate ultrasound images, e.g., by ultrasound probe 106, computing devices 102, or another device or system. In some examples, ultrasound probe 106 is a linear array ultrasound probe that detects returning ultrasound waves along a single plane oriented orthogonal to the direction of travel of the ultrasonic waves. A linear array ultrasound probe may generate 2D ultrasound images. In some examples, ultrasound probe 106 may be configured to perform 3D ultrasound, e.g., by rotating a linear array of ultrasound transducers.

MR visualization device 104 may use various visualization techniques to display MR visualizations to clinician 110. A MR visualization may comprise one or more virtual objects that are viewable by a user at the same time as real-world objects. Thus, what clinician 110 sees is a mixture of real and virtual objects.

MR visualization device 104 may comprise various types of devices for presenting MR visualizations. For instance, in some examples, MR visualization device 104 may be a Microsoft HOLOLENS™ headset, such as the HOLOLENS 2 headset, available from Microsoft Corporation, of Redmond, Washington, USA, or a similar device, such as, for example, a similar MR visualization device that includes waveguides. The HOLOLENS™ device can be used to present 3D virtual objects via holographic lenses, or waveguides, while permitting a user to view actual objects in a real-world scene, i.e., in a real-world environment, through the holographic lenses. In some examples, MR visualization device 104 may be a holographic projector, head-mounted smartphone, special-purpose MR visualization device, or another type of device for presenting MR visualizations. In some examples, MR visualization device 104 includes a head-mounted unit and a backpack unit that performs at least some of the processing functionality of MR visualization device 104. In other examples, all functionality of MR visualization device 104 is performed by hardware residing in a head-mounted unit. Discussion in this disclosure of actions performed by system 100 may be performed by one or more computing devices 102 of system 100, MR visualization device 104, or a combination of the one or more computing devices and MR visualization device 104.

Processing circuitry performing computing tasks of system 100 may be distributed among one or more of computing devices 102, MR visualization device 104, ultrasound probe 106, and/or other computing devices. Furthermore, in some examples, system 100 may include multiple MR visualization devices. Computing devices 102 may include server computers, personal computers, smartphones, tablet computers, laptop computers, and other types of computing devices. Computing devices 102 may communicate with MR visualization device 104 via one or more wired or wireless communication links In the example of FIG. 1, a lightning bolt 118 represents a wireless communication link between computing devices 102 and MR visualization device 104.

In accordance with one or more techniques of this disclosure, system 100 may obtain reference data depicting one or more bones of patient 112. Medical imaging system 108 may generate the reference data. Medical imaging system 108 may generate the reference data prior to the ultrasound examination. In some examples, medical imaging system 108 generates computed tomography (CT) data. In other examples, medical imaging system 108 may generate magnetic resonance imaging (MRI) data or other types of medical images.

Furthermore, system 100 may determine a spatial relationship between ultrasound probe 106 and the bone. In other words, system 100 may determine where ultrasound probe 106 is in relation to the actual bone of patient 112. System 100 may determine this spatial relationship based on the reference data and ultrasound data generated by ultrasound probe 106. Ultrasound probe 106 generates the ultrasound data during use of ultrasound probe 106 on patient 112. The ultrasound data may include an ultrasound image or system 100 may generate an ultrasound image based on the ultrasound data generated by ultrasound probe 106.

As part of determining the spatial relationship between ultrasound probe 106 and the bone, system 100 may determine a current physical location of ultrasound probe 106. The current physical location of ultrasound probe 106 may be expressed in terms of coordinates in a real-world coordinate system. The real-world coordinate system may express positions within a physical environment of patient 112. In some examples, system 100 uses data from one or more sensors (e.g., depth sensors, visible light sensors, etc.) included in MR visualization device 104 to determine the current physical location of ultrasound probe 106. In some examples, system 100 may use data from one or more other sensors in an examination room to determine the current physical location of ultrasound probe 106. In some examples, one or more markers attached to ultrasound probe 106 help system 100 determine the current physical location of ultrasound probe 106.

In some examples, to determine the spatial relationship between ultrasound probe 106 and the bone, system 100 may obtain one or more ultrasound images based on the ultrasound data generated by ultrasound probe 106. The ultrasound image may represent structures within patient 112 in a slice aligned with a detection plane (or axis) of ultrasound probe 106. In general, a transducer of ultrasound probe 106 emits pulses of ultrasonic waves onto the skin of patient 112. In some examples, a gel may be applied to the skin of patient 112 to increase transmission of the ultrasonic waves generated by ultrasound probe 106 into the interior of patient 112. When a pulse of ultrasonic waves encounters a first structure (e.g., muscle, tendon, ligament, blood vessel, cartilage, bone, etc.) within patient 112, the first structure may reflect a portion of the ultrasonic waves of the pulse back toward the transducer of ultrasound probe 106, which may then detect the reflected portion of the ultrasonic waves. However, the first structure may also transmit a portion of the ultrasonic waves of the pulse through the first structure. A second structure may reflect a portion of the ultrasonic waves of the pulse that were transmitted through the first structure and may transmit another portion of the ultrasonic waves of the pulse, and so on. Based on one or more estimated speeds of travel of the ultrasonic waves through patient 112 and based on a time required for the ultrasonic waves reflected by a structure to arrive back at the transducer of ultrasound probe 106, the distance of the structure from the transducer of ultrasound probe 106 may be estimated.

System 100 may obtain an ultrasound image based on estimated distances to structures within patient 112. For instance, the ultrasound image may include pixels corresponding to distances from a transducer of ultrasound probe 106. In a typical ultrasound image, pixels corresponding to distances of structures that reflect ultrasonic waves are shown in white while other pixels remain dark.

In an example where ultrasound probe 106 is a linear array ultrasound probe, ultrasound probe 106 includes an array of transducers arranged in a single line along the detection plane of ultrasound probe 106. The transducers may be arranged in a fan shape. Thus, an ultrasound image generated by a linear array ultrasound probe may represent structures within a fan-shaped slice through patient 112 aligned with the detection plane. In some examples, a 3D ultrasound image of a cone-shaped section of patient 112 may be generated by rotating the linear array of transducers of ultrasound probe 106.

The structures represented in the ultrasound image may include soft tissue structures and bone. System 100 may analyze the ultrasound image to identify a structure represented in the ultrasound image that has the same profile as a bone represented in the reference data. For instance, system 100 may analyze the ultrasound image to identify a curve of a structure represented in the ultrasound image. System 100 may then attempt to match that curve to a curve of a bone represented in the reference data. If system 100 finds a match, the structure represented in the ultrasound image is likely to be the bone represented in the reference data.

Moreover, if system 100 finds the match, system 100 may determine real-world coordinates for the bone. System 100 may determine the real-world coordinates of the bone based on the distance of the bone from ultrasound probe 106 (as determined using the ultrasound image) and the real-world coordinates of ultrasound probe 106. Points on the bone as depicted in the reference data may be defined by a virtual coordinate system. Because system 100 is able to match a curve of the bone represented in the reference data with a curve of the bone represented in the ultrasound image, system 100 is therefore able to determine a relationship between the virtual coordinate system of the reference data and the real-world coordinate system. In other words, system 100 may generate registration data that registers the reference data with the real-world coordinate system.

After registering the reference data with the real-world coordinate system, system 100 may generate virtual guidance based on the reference data, the registration data, the physical location of ultrasound probe 106. The virtual guidance may provide guidance to clinician 110 regarding how ultrasound probe 106 is positioned relative to a target position at which ultrasound probe 106 is able to generate ultrasound data that provides information regarding a soft tissue structure of the patient. For example, the virtual guidance may instruct clinician 110 how to move ultrasound probe 106 so that ultrasound probe 106 is at a target position to generate ultrasound data that provides information regarding a soft tissue structure of patient 112. In some examples, the virtual guidance may provide clinician 110 with information that ultrasound probe 106 is currently positioned at the target position. System 100 may then cause a MR visualization device 104 to output the virtual guidance to clinician 110.

System 100 may generate various types of virtual guidance. For example, clinician 110 may be preparing for a shoulder replacement surgery. In this example, clinician 110 may need to take the properties of various soft tissue structures into account when determining how to select and implant a glenoid prosthesis and/or humeral prosthesis. For example, laxity in the rotator cuff muscles (e.g., the supraspinatus muscle, infraspinatus muscle, teres minor muscle, and subscapularis muscle) may suggest the use of a reverse total shoulder arthroplasty instead of an anatomic total shoulder arthroplasty Accordingly, in this example, it may be valuable for clinician 110 to understand the locations and sizes of the rotator cuff muscles. A single ultrasound image that represents a 2D slice through patient 112 may show an edge of a rotator cuff muscle but might not show enough of the entire rotator cuff muscle to allow clinician 110 to understand the location and size of the rotator cuff muscle of patient 112. Accordingly, in this example, the virtual guidance generated by system 100 may instruct clinician 110 how to move ultrasound probe 106 to one or more positions where ultrasound probe 106 can generate ultrasound data that provides more information regarding the rotator cuff muscle of patient 112.

As noted above, system 100 may generate the virtual guidance based on the reference data. In general, the reference data provides a more complete and precise representation of bones than may be generated by ultrasound probe 106. In general, system 100 can predict the positions of various soft tissue structures based on the shapes and positions of the bones represented in the reference data. Thus, because both the reference data and ultrasound probe 106 are registered with the real-world coordinate system, system 100 may generate the virtual guidance that provides guidance to clinician 110 regard how ultrasound probe 106 is positioned relative to a target position at which ultrasound probe 106 is able to generate ultrasound data that provides information regarding a soft tissue structure of patient 112. For instance, the virtual guidance may instruct clinician 110 to move ultrasound probe 106 to a predicted location of the soft tissue structure. System 100 may update the virtual guidance as clinician 110 moves ultrasound probe 106 from position to position. Thus, clinician 110 may obtain real-time feedback on how to move ultrasound probe 106 so that ultrasound probe 106 is able to generate the ultrasound data. In this way, ultrasound probe 106 may generate ultrasound data regarding the portion of the soft tissue structure relevant to clinician 110.

In some examples, system 100 may generate a virtual model of a soft tissue structure of patient 112 based on ultrasound data regarding the soft tissue structure. For instance, in one example, the virtual guidance may instruct clinician 110 to slide ultrasound probe 106 along the skin of patient 112 over the predicted location of the soft tissue structure. System 100 may obtain a series of ultrasound images based on ultrasound data generated by ultrasound probe 106 as clinician 110 slides ultrasound probe 106 over the predicted location of the soft tissue structure. System 100 may segment the ultrasound images to isolate parts of the ultrasound images that correspond to the soft tissue structure. In some examples, system 100 may use a machine learning (ML) based computer vision technique (e.g., a convolutional neural network) to segment the ultrasound images to isolate the parts of the ultrasound images that correspond to the soft tissue structure. System 100 may then process the parts of the ultrasound images that correspond to the soft tissue structure to form the virtual model of the soft tissue structure.

MR visualization device 104 may output the virtual model of the soft tissue structure so that the virtual model of the soft tissue structure appears to clinician 110 to be superimposed on patient 112 at an actual location of the soft tissue structure. MR visualization device 104 may also output virtual models of one or more bones of patient 112 so that the virtual bones of the bones appear to clinician 110 to be superimposed on patient 112 at actual locations of the bones of patient 112. In this way, clinician 110 may be able to easily comprehend the locations of hidden soft tissue structures and bones of patient 112. Being able to view virtual models of the soft tissue structure and bones on MR visualization device 104 may be especially valuable during a surgery.

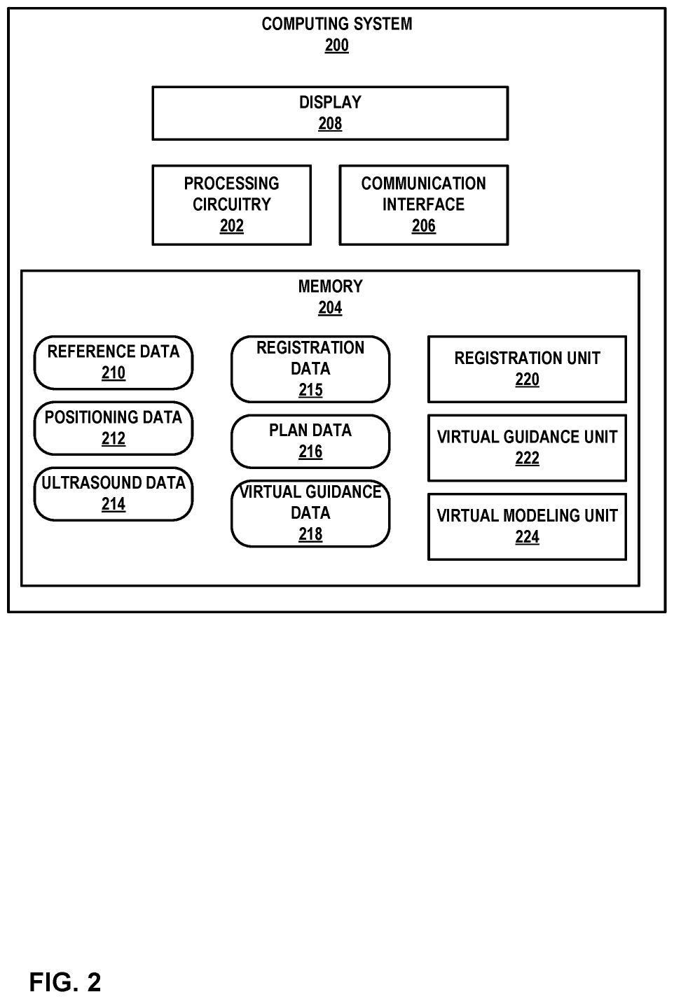

FIG. 2 is a conceptual diagram illustrating an example computing system 200 in accordance with one or more techniques of this disclosure. Components of computing system 200 of FIG. 2 may be included in one of computing devices 102 (FIG. 1), MR visualization device 104, or ultrasound probe 106. In the example of FIG. 2, computing system 200 includes processing circuitry 202, memory 204, a communication interface 206, and a display 208.

Examples of processing circuitry 202 include one or more microprocessors, digital signal processors (DSPs), application specific integrated circuits (ASICs), field programmable gate arrays (FPGAs), hardware, or any combinations thereof. In general, processing circuitry 202 may be implemented as fixed-function circuits, programmable circuits, or a combination thereof. Fixed-function circuits refer to circuits that provide particular functionality and are preset on the operations that can be performed. Programmable circuits refer to circuits that can programmed to perform various tasks and provide flexible functionality in the operations that can be performed. For instance, programmable circuits may execute software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. In some examples, one or more of the units may be distinct circuit blocks (fixed-function or programmable), and in some examples, the one or more units may be integrated circuits.

Processing circuitry 202 may include arithmetic logic units (ALUs), elementary function units (EFUs), digital circuits, analog circuits, and/or programmable cores, formed from programmable circuits. In examples where the operations of processing circuitry 202 are performed using software executed by the programmable circuits, memory 204 may store the object code of the software that processing circuitry 202 receives and executes, or another memory within processing circuitry 202 (not shown) may store such instructions. Examples of the software include software designed for surgical planning. Processing circuitry 202 may perform the actions ascribed in this disclosure to computing system 200.

Memory 204 may store various types of data used by processing circuitry 202. Memory 204 may include any of a variety of memory devices, such as dynamic random access memory (DRAM), including synchronous DRAM (SDRAM), magnetoresistive RAM (MRAM), resistive RAM (RRAM), or other types of memory devices. Examples of display 208 include a liquid crystal display (LCD), a plasma display, an organic light emitting diode (OLED) display, or another type of display device.

Communication interface 206 that allows computing system 200 to output data and instructions to and receive data and instructions from MR visualization device 104, medical imaging system 108, or other device via one or more communication links or networks. Communication interface 206 may be hardware circuitry that enables computing system 200 to communicate (e.g., wirelessly or using wires) to other computing systems and devices, such as MR visualization device 104. Example networks may include various types of communication networks including one or more wide-area networks, such as the Internet, local area networks, and so on. In some examples, the network may include wired and/or wireless communication links.

In the example of FIG. 2, memory 204 stores reference data 210, positioning data 212, ultrasound data 214, registration data 215, plan data 216, and virtual guidance data 218. Additionally, in the example of FIG. 2, memory 204 stores a registration unit 220, a virtual guidance unit 222, and a virtual modeling unit 224. In other examples, memory 204 may store more, fewer, or different types of data or units. Moreover, the data and units illustrated in the example of FIG. 2 are provided for purposes of explanation and may not represent how data is actually stored or how software is actually implemented. Registration unit 220, virtual guidance unit 222, and virtual modeling unit 224 may comprise instructions that are executable by processing circuitry 202. For ease of explanation, this disclosure may describe registration unit 220, virtual guidance unit 222, virtual modeling unit 224 as performing various actions when processing circuitry 202 executes instructions of registration unit 220, virtual guidance unit 222, virtual modeling unit 224.

In general, reference data 210 includes previously obtained data depicting one or more bones of patient 112. For example, reference data 210 may include one or more CT images of a bone. In some examples, reference data 210 may include a 3-dimensional model of a bone. The 3-dimensional model of the bone may be generated based on a plurality of CT images. Computing system 200 may obtain reference data 210 from medical imaging system 108 or another source. For instance, computing system 200 may generate reference data 210 based on data received from medical imaging system 108 or another source; or computing system 200 may receive reference data 210 from medical imaging system 108 or another source.

Positioning data 212 may include data indicating locations of ultrasound probe 106, patient 112, and/or other real-world objects. Computing system 200 may obtain positioning data 212 based on one or more sensors, such as depth sensors or cameras, located on MR visualization device 104 and/or other devices. Ultrasound data 214 may include ultrasound images or other types of data generated by ultrasound probe 106. In some examples, computing system 200 may use the data generated by ultrasound probe 106 to generate ultrasound images. Plan data 216 may include data related to a plan for a medical task. For instance, plan data 216 may indicate which soft tissue structures are relevant for the medical task.

As described in greater detail elsewhere in this application, registration unit 220 may determine a physical location of ultrasound probe 106. Additionally, registration unit 220 may generate, based on first ultrasound data generated by ultrasound probe 106, registration data that registers virtual locations on the bone of patient 112 as depicted in reference data 210 with corresponding physical locations on the bone of patient 112. Virtual guidance unit 222 may generate virtual guidance data 218 based on reference data 210, registration data 215, and the physical location of ultrasound probe 106 (e.g., positioning data 212). Virtual guidance data 218 may provide guidance to clinician 110 regarding how ultrasound probe 106 is positioned relative to a target position at which ultrasound probe 106 is able to generate ultrasound data that provides information regarding a soft tissue structure of the patient. For example, virtual guidance data 218 may instruct clinician 110 how to move ultrasound probe 106 so that ultrasound probe 106 is at a target position to generate ultrasound data that provides information regarding a soft tissue structure of patient 112. Virtual guidance unit 222 may cause MR visualization device 104 to output the virtual guidance to clinician 110. Virtual modeling unit 224 may generate virtual models and, in some examples, may cause MR visualization device 104 to output the virtual models.

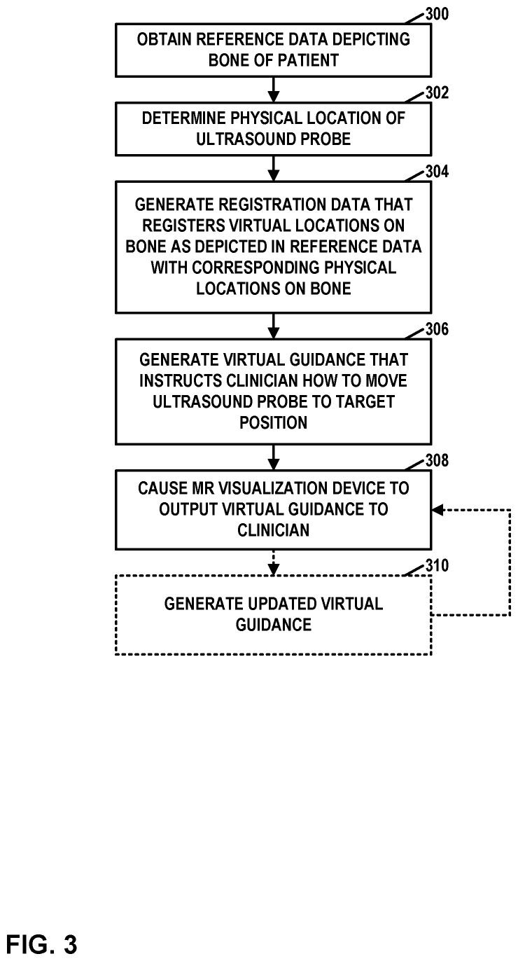

FIG. 3 is a flowchart illustrating an example operation of system 100 in accordance with one or more techniques of this disclosure. The flowcharts of this disclosure illustrate example operations. In other examples, operations may include more, fewer, or different actions.

In the example of FIG. 3, computing system 200 may obtain reference data 210 depicting at least one bone of patient 112 (300). As described elsewhere in this disclosure, computing system 200 may obtain reference data 210 from medical imaging system 108 or another source.

Furthermore, registration unit 220 may determine a physical location of ultrasound probe 106 (302). In some examples, registration unit 220 may determine the physical location of ultrasound probe 106 based on data from one or more sensors of MR visualization device 104. For instance, MR visualization device 104 may include one or more visible-light cameras and a depth sensor. The depth sensor may be configured to detect a distance from the depth sensor to an object, such as ultrasound probe 106. The depth sensor may be implemented in one of a variety of ways. For instance, the depth sensor may include an infrared light emitter and detector. The infrared light emitter may emit pulses of infrared light. Reflections of the infrared light are detected by the detector of the depth sensor. The depth sensor may determine, based on a time-of-flight of the pulse of infrared light to an object and back to the detector from the object, a distance from the depth sensor to the object. In some examples, registration unit 220 may be configured to use signals from the visible light sensors to identify ultrasound probe 106. In some examples, optical markers may be attached to ultrasound probe 106 to enhance the ability of registration unit 220 to identify ultrasound probe 106 based on the signals from the visible light sensors of MR visualization device 104. Determining the location of ultrasound probe 106 based on data from sensors of MR visualization device 104, as opposed to other types of devices, may be advantageous because use of data from sensors of MR visualization device 104 may eliminate the need for another object to be in a surgical theater that may need to be sterilized or otherwise shielded. Moreover, use of data from sensors of MR visualization device 104 may be advantageous because the sensors of MR visualization device 104 may detect ultrasound probe 106 from the perspective of clinician 110 using ultrasound probe 106. Therefore, clinician 110 is not blocking the view of ultrasound probe 106 from other sensors.

Registration unit 220 may indicate the physical location of ultrasound probe 106 in terms of coordinates in a real-world coordinate system. The real-world coordinate system may be a coordinate system describing locations of objects in a physical environment of MR visualization device 104 and patient 112. MR visualization device 104 may establish the real-world coordinate system by performing a Simultaneous Localization and Mapping (SLAM) algorithm. The SLAM algorithm also determines a current position of MR visualization device 104 in terms of the real-world coordinate system.

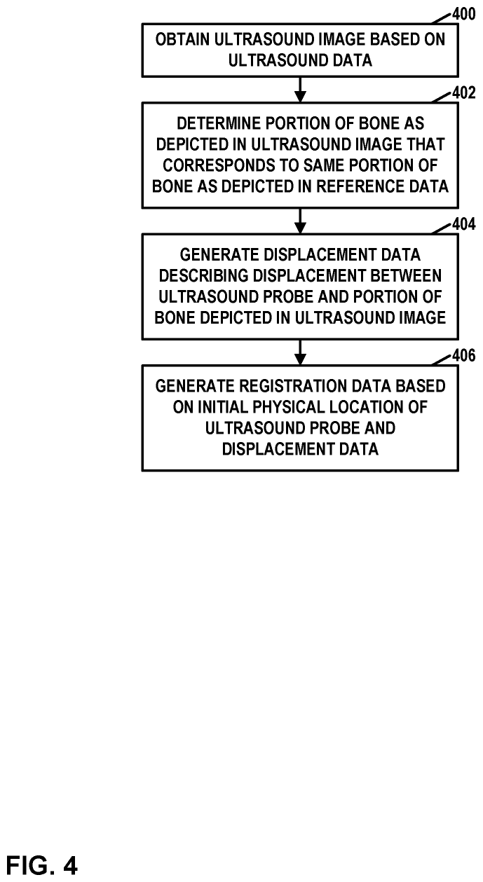

Registration unit 220 may generate, based on ultrasound data generated by ultrasound probe 106, registration data that registers virtual locations on the bone of patient 112 as depicted in reference data 210 with corresponding physical locations on the bone of patient 112 (304). Registration unit 220 may generate the registration data in one of a variety of ways. For instance, FIG. 4, which is described in greater detail elsewhere in this disclosure, is a flowchart illustrating an example operation of the system for generating registration data.

Furthermore. virtual guidance unit 222 may generate virtual guidance based on reference data 210, registration data 215, and the physical location of ultrasound probe 106 (306). The virtual guidance may provide guidance to clinician 110 regarding how ultrasound probe 106 is positioned relative to a target position at which ultrasound probe 106 is able to generate ultrasound data that provides information regarding a soft tissue structure of the patient. For example, the virtual guidance may instruct clinician 110 how to move ultrasound probe 10 so that ultrasound probe 106 is at a target position to generate ultrasound data that provides information regarding a soft tissue structure of patient 112.

Plan data 216 (FIG. 2) may include data information describing a plan for clinician 110 to follow with respect to patient 112. In some examples, plan data 216 may include surgical planning data that describe a process to prepare for and conduct a surgery on patient 112. In some examples, plan data 216 may be limited to just an ultrasound examination of patient 112. In either case, plan data 216 may indicate which soft tissue structures are to be scanned during the ultrasound examination. For instance, plan data 216 may indicate that a supraspinatus muscle is to be scanned during the ultrasound examination. On the basis of plan data 216 indicating that the soft tissue structure is to be scanned during the ultrasound examination, virtual modeling unit 224 may obtain (e.g., generate or receive) an estimated model of the soft tissue structure based on the reference data. For example, virtual modeling unit 224 may use a statistical shape model of the bone as depicted in reference data 210 as a basis for the estimated model of the soft tissue structure. In other words, virtual guidance unit 222 may generate the estimated model of the soft tissue structure as a statistical shape model (SSM) of the soft tissue structure based on reference data 210. In general terms, when virtual modeling unit 224 uses a statistical shape model to generate the estimated model of the soft tissue structure, virtual modeling unit 224 may use statistics regarding the bone to determine an expected size and shape of the soft tissue structure.

In some examples, the statistical shape model is implemented using a machine learning (ML) model. For instance, in an example where the ML model is a neural network, virtual modeling unit 224 may train the neural network to generate the estimated model of the soft tissue structure (or other data sufficient to characterize the soft tissue structure) as output. Input to the neural network may include information regarding one or more bones (e.g., models of the bones, data characterizing the one or more bones), patient demographic data, and/or other types of data. The neural network may be trained based on data from many people. Accordingly, the estimated model of the soft tissue structure generated by the neural network may be considered to be a prediction of the soft tissue structure given the corresponding soft tissue structure and bones of many other people.

Additionally, virtual guidance unit 222 may generate, based on the registration data that registers virtual locations on the bone of patient 112 as depicted in reference data 210 with corresponding physical locations on the bone of patient 112, additional registration data (e.g., second registration data) that registers virtual locations on the estimated model of the soft tissue structure and corresponding physical locations on the bone. For example, virtual guidance unit 222 may determine a location on the bone as depicted in reference data 210 of an expected attachment point of the soft tissue structure to the bone. Additionally, in this example, virtual guidance unit 222 may determine in the estimated model of the soft tissue structure corresponding attachment points of the soft tissue structure to the bone. Because virtual guidance unit 222 has first registration data that registers virtual locations on the bone of patient 112 as depicted in reference data 210 with corresponding physical locations on the bone of patient 112, virtual guidance unit 222 is therefore able to determine how a virtual coordinate system by which positions on the estimated model of the soft tissue structure relate to the virtual locations on the bone of patient 112 as depicted in reference data 210, and therefore how the virtual coordinate system by which positions on the estimated model of the soft tissue structure relate to the real-world coordinate system (i.e., the physical locations on the bone of the patient).

Furthermore, virtual guidance unit 222 may determine, based on the additional registration data and the physical location of ultrasound probe 106, a direction to move ultrasound probe 106 so that ultrasound probe 106 is at the target position. In some examples, the direction may be a lateral movement of ultrasound probe 106 across the skin of patient 112. In some examples, the direction may be a rotation of ultrasound probe 106. In some examples, the direction may be a change of angle of ultrasound probe 106 relative to the surface of the skin of patient 112.

In some examples, to generate the virtual guidance, virtual guidance unit 222 may track which parts of the soft tissue structure have been scanned during the ultrasound examination. For instance, virtual guidance unit 222 may determine which surfaces of the estimated model of the soft tissue structure have not yet been within the detection plane of ultrasound probe 106. Virtual guidance unit 222 may then generate the virtual guidance to direct clinician 110 so that ultrasound probe 106 is positioned such that an unscanned part of the soft tissue structure is within the detection plane of ultrasound probe 106. Therefore, when sufficient parts of the soft tissue structure have been scanned, the ultrasound examination of the soft tissue structure may be complete. Note that in order to scan some part of the soft tissue structure, virtual guidance unit 222 may generate virtual guidance that instructs clinician 110 to rotate or tilt ultrasound probe 106. Thus, in some examples, the virtual guidance that instructs clinician 110 how to move ultrasound probe 106 indicates how to adjust an angle of ultrasound probe 106 relative to patient 112 so that ultrasound probe 106 is in the target position to generate additional ultrasound data. In some examples, the virtual guidance may indicate to clinician 110 that ultrasound probe 106 is at a correct angle to generate the additional ultrasound data.

Additionally, virtual guidance unit 222 may cause MR visualization device 104 to output the virtual guidance to clinician 110 (308). For example, virtual guidance unit 222 may send signals to MR visualization device 104 that instruct MR visualization device 104 to display the virtual guidance. In some examples, virtual guidance unit 222 may cause MR visualization device 104 to output the virtual guidance so that the virtual guidance appears to clinician 110 to be superimposed on patient 112.

In some examples, virtual guidance unit 222 may generate updated virtual guidance (310). The updated virtual guidance may instruct clinician 110 to move ultrasound probe 106 to a next target position so that ultrasound probe 106 can generate additional ultrasound data regarding the soft tissue structure or a different soft tissue structure. In some examples, the updated virtual guidance may indicate to clinician 110 that ultrasound probe 106 is not yet at the next target position. Virtual guidance unit 222 may then cause MR visualization device 104 to display the updated virtual guidance (308). This process may continue until ultrasound probe 106 generates sufficient ultrasound data. In this way, if the virtual guidance is considered first virtual guidance, virtual guidance unit 222 may obtain second ultrasound data and determine second virtual guidance based on the reference data, the registration data, and the physical location of ultrasound probe 106. In some examples, the second virtual guidance may instruct clinician 110 how to move ultrasound probe 106 so that ultrasound probe 106 is in a second target position to generate third ultrasound data that provides additional information regarding the soft tissue structure of patient 112. In some examples, the second virtual guidance may indicate whether ultrasound probe 106 is at the second target position. Virtual guidance unit 222 may then cause the MR visualization device to output the second virtual guidance to clinician 110.

Virtual guidance unit 222 may generate the updated virtual guidance based on second ultrasound data generated by ultrasound probe 106 when ultrasound probe 106 is at the target position. For example, virtual guidance unit 222 may refine an estimated model of the soft tissue structure based on the ultrasound data generated by ultrasound probe 106 when ultrasound probe 106 is at the target position. In this example, virtual guidance unit 222 may generate the updated virtual guidance based on the refined estimated model.

Virtual guidance unit 222 may refine the estimated model in various ways. For example, virtual guidance unit 222 may implement a machine learning (ML) model, such as an artificial neural network. Inputs to the ML model may include data representing a 3D model of the soft tissue structure and data derived from the ultrasound data. An initial 3D model of the soft tissue structure may be generated using a statistical shape model based on the reference data and, in some examples, other factors such as the age, sex, weight, and other characteristics of patient 112. Outputs of the ML model may include data representing an updated 3D model of the soft tissue structure. The data derived from the ultrasound data may include data indicating a measured position of the soft tissue structure, thickness of the soft tissue structure, density of the soft tissue structure, and other types of information that system 100 can derive from the second ultrasound data. Subsequently, when virtual guidance unit 222 obtains more new ultrasound data, virtual guidance unit 222 may use the updated 3D model of the soft tissue structure, as well as data based on the new ultrasound data, as input to the artificial neural network. The artificial neural network may be various types of artificial neural networks, such as a convolutional neural network or fully connected deep neural network. In some examples, virtual guidance unit 222 may use image stitching techniques to detect the boundaries between acquired ultrasound images. In some examples, virtual guidance unit 222 may use feature-based detectors to detect features that are shared among ultrasound images. Example feature-based detectors include Scale Invariant Feature Transform (SIFT), Speeded Up Robust Features (SURF), Pyramidal Histogram of Visual Words (PHOW).

FIG. 4 is a flowchart illustrating an example operation of computing system 200 for generating registration data, in accordance with one or more techniques of this disclosure. In the example of FIG. 4, registration unit 220 may obtain an ultrasound image based on ultrasound data (400). Ultrasound probe 106 may generate the ultrasound data while ultrasound probe 106 is at an initial physical location.

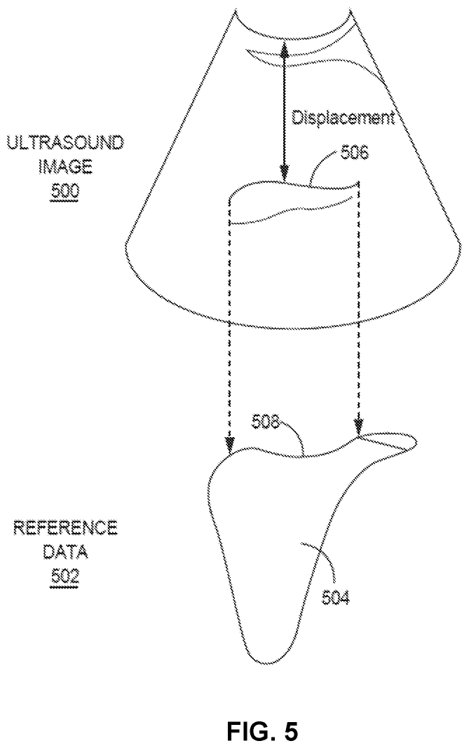

Additionally, registration unit 220 may determine a portion of the bone as depicted in the ultrasound image that corresponds to a portion of the bone as depicted in reference data 210 (402). In some examples, to determine the portion of the bone as depicted in the ultrasound image that corresponds to the portion of the bone as depicted in reference data 210, registration unit 220 may perform a curve-matching process As example of the curve-matching process is described below with respect to the example of FIG. 5. In other examples, registration unit 220 may use deep learning or a convolution neural network to perform the curve matching process In some examples, registration unit 220 may use a wavelet transform to determine feature vectors that characterize textures in the ultrasound images. Registration unit 220 may form elements of the feature vectors by wavelet transformations at one or more decomposition levels. Registration unit 220 may implement a classifier that may use the feature vector to recognize structures in different ultrasound images.

Furthermore, in the example of FIG. 4, registration unit 220 may generate displacement data describing a spatial displacement between ultrasound probe 106 and the portion of the bone depicted in the ultrasound image (404). For example, registration unit 220 may generate a displacement vector that includes components indicating a displacement, in the detection plane of ultrasound probe 106, between a transducer of ultrasound probe 106 and a location on the portion of the bone that reflected ultrasonic waves back to the transducer. In some examples, the components may include a distance value and an angle value indicating an angle of the transducer relative to a midline of an array of transducers of ultrasound probe 106. In some examples, the components may include a first value indicating a displacement of the location on the bone along a line orthogonal to the midline of the array of transducers of ultrasound probe 106 and a second value indicating a displacement of the location on the bone along the midline of the array of transducers of ultrasound probe 106.

Registration unit 220 may generate the registration data based on the initial physical location of ultrasound probe 106 and the displacement data (406). For example, the initial physical location of ultrasound probe 106 may be represented in terms of real-world coordinates. In this example, the displacement data may also be expressed or converted to real-world coordinates. Thus, the location on the bone may be expressed in terms of real-world coordinates by adding the real-world coordinates of ultrasound probe 106 and the displacement data. Furthermore, registration unit 220 may determine the virtual coordinates (i.e., coordinates defining positions in the registration data) of the corresponding location on the bone in the reference data. Therefore, registration unit 220 may generate the registration data by determining the relationship between the real-world coordinates of the location on the bone and the virtual coordinates of the corresponding location on the bone in the reference data.

FIG. 5 is a conceptual diagram illustrating matching curves in accordance with one or more techniques of this disclosure. The example of FIG. 5 shows an ultrasound image 500 and reference data 502. Reference data 502 includes a reference model 504 of a scapula of patient 112. Reference model 504 may be a 3D dimensional model of the scapula. Although FIG. 5 is described with respect to a scapula, the processes described with respect to FIG. 5 may be applicable to other bones, such as a pelvis, humerus, tibia, fibula, femur, patella, radius, ulna, talus, metatarsal, phalange, cuneiform bones, cuboid bone, calcaneus, carpal bone, and so on.

To determine a portion of a bone (e.g., scapula) as depicted in ultrasound image 500 that corresponds to the portion of the bone as depicted in reference data 502, registration unit 220 may generate curve data that characterizes a curve 506 of the bone as depicted in ultrasound image 500. Curve 506 may correspond to an outer surface of the bone as viewed along the detection plane of ultrasound probe 106. Registration unit 220 may then search the bone as depicted in reference data 502 for a curve that matches the curve of the bone as depicted in ultrasound image 500. In other words, registration unit 220 may analyze reference data 502 to identify a curve that matches the curve of the bone as depicted in ultrasound image 500.

To generate curve data, registration unit 220 may apply an edge detection algorithm to ultrasound image 500. The edge detection algorithm detects edge in ultrasound image 500. Registration unit 220 may apply one or more of a variety of known edge detection algorithms, such as the Canny edge detector, a second-order edge detector, or another edge detection algorithm. Registration unit 220 may then perform curve fitting on the detected edges; for instance, registration unit 220 may perform a polynomial regression or other type of regression to perform curve fitting. Additionally, registration unit 220 may perform curve fitting on surfaces of reference model 504 taken along multiple slices passing at a plurality of angles through the reference model. As part of searching the bone as depicted in registration reference data 502 for a curve that matches the curve of the bone as depicted in ultrasound image 500, registration unit 220 may compare curve 506 to curves of surfaces of reference model 504. For instance, registration unit 220 may compare the coefficients of polynomial functions generated by performing polynomial regression on curve 506 and the curves of the surfaces of reference model 504. In the example of FIG. 5, registration unit 220 may determine that curve 508 on reference model 504 corresponds to curve 506 in ultrasound image 500.

Because curves 506 and 508 are lines and not single points, registration unit 220 may, therefore determine the rotation of reference model 504 relative to the bone depicted in ultrasound image 500. Registration unit 220 may use the rotation of reference model 504 relative to the bone depicted in ultrasound image 500 as part of generating the registration data that registers a virtual location on the bone of patient 112 as depicted in ultrasound image 500 with a corresponding physical location on the bone of patient 112. In some examples, generating the registration data includes generating a transform matrix.

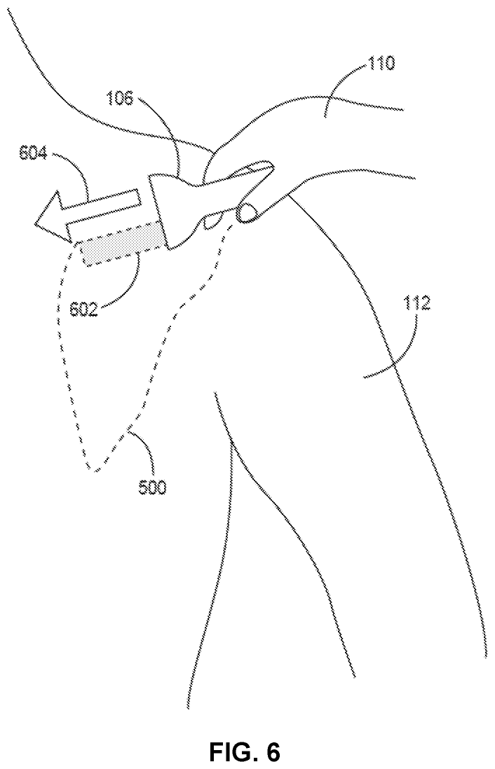

FIG. 6 is a conceptual diagram illustrating example virtual guidance during an ultrasound examination of a shoulder of patient 112, in accordance with one or more techniques of this disclosure. In the example of FIG. 6, clinician 110 holds ultrasound probe 106 against the skin of patient 112. In the example of FIG. 6, only a hand of clinician 110 is shown.

MR visualization device 104 (not shown in the example of FIG. 6) displays a scapula model 600 that represents a scapula of patient 112. Scapula model 600 is a virtual model and is positioned at a location corresponding to the actual scapula of patient 112. Virtual modeling unit 224 (FIG. 2) may generate scapula model 600 based on reference data depicting the scapula of patient 112.

Additionally, MR visualization device 104 displays a supraspinatus model 602 that represents a supraspinatus muscle of patient 112. Supraspinatus model 602 is a virtual model and is positioned at a location corresponding to the actual supraspinatus muscle of patient 112. Virtual modeling unit 224 may generate supraspinatus model 602 based on reference data 210. For instance, virtual modeling unit 224 may use parameters of bones depicted in reference data 210 to perform a statistical shape modeling process that generates supraspinatus model 602. Presentation of supraspinatus model 602 may be a type of virtual guidance. In some examples, virtual modeling unit 224 may refine supraspinatus model 602 based on ultrasound data 214 generated by ultrasound probe 106, e.g., as described elsewhere in this disclosure. In this way, virtual guidance unit 222 may cause MR visualization device 104 to output a model of the bone and a model of the soft tissue structure so that the model of the bone and the model of the soft tissue structure appear to the clinician to be superimposed on the patient

MR visualization device 104 may display a virtual directional element 604 that indicates how clinician 110 is to move ultrasound probe 106. For instance, virtual directional element 604 may indicate how clinician 110 is to move ultrasound probe 106 to generate ultrasound data that provides more information about the supraspinatus muscle of patient 112. Specifically, in the example of FIG. 6, virtual directional element 604 indicates that clinician 110 is to move ultrasound probe 106 medially. Furthermore, as shown in the example of FIG. 6, MR visualization device 104 may display virtual directional clement 604 (or other virtual guidance) so that virtual directional element 604 appears to clinician 110 to be superimposed on patient 112. Displaying virtual directional element 604 (and/or other virtual guidance) superimposed on patient 112 may make it easier for clinician 110 to understand how to move ultrasound probe 106. In other examples, MR visualization device 104 may display virtual directional element 604 (or other virtual guidance) at another location in a field of view of clinician 110.

In some examples, the virtual guidance that instructs clinician 110 how to move ultrasound probe 106 may instruct clinician 110 to move ultrasound probe 106 laterally across the skin of patient 112, e.g., as shown in the example of FIG. 6. In some examples, the virtual guidance that instructs clinician 110 how to move ultrasound probe 106 may instruct clinician 110 to rotate ultrasound probe 106. In some examples, the virtual guidance that instructs clinician 110 how to move ultrasound probe 106 may instruct clinician 110 to change an angle at which ultrasound probe 106 contacts the skin of patient 112. Changing the rotation angle or skin-contact angle of ultrasound probe 106 may enable ultrasound probe 106 to gather more information about internal structures of patient 112.

The following is a non-limiting set of examples that are in accordance with one or more techniques of this disclosure.
Aspect 1: A method includes obtaining reference data depicting a bone of a patient; determining a physical location of an ultrasound probe; generating, based on first ultrasound data generated by the ultrasound probe, registration data that registers virtual locations on the bone of the patient as depicted in the reference data with corresponding physical locations on the bone of the patient; generating virtual guidance based on the reference data, the registration data, and the physical location of the ultrasound probe, wherein the virtual guidance provides guidance to a clinician regarding how the ultrasound probe is positioned relative to a target position at which the ultrasound probe is able to generate second ultrasound data that provides information regarding a soft tissue structure of the patient; and causing a bead-mounted Mixed Reality (MR) visualization device to output the virtual guidance to the clinician.
Aspect 2: The method of aspect 1, wherein: the physical location of the ultrasound probe is a physical location at which the ultrasound probe generated the first ultrasound data, and generating the registration data comprises: obtaining an ultrasound image based on the first ultrasound data; determining a portion of the bone as depicted in the ultrasound image that corresponds to a portion of the bone as depicted in the reference data; generating displacement data describing a spatial displacement between the ultrasound probe and the portion of the bone depicted in the ultrasound image; and generating the registration data based on the physical location of the ultrasound probe and the displacement data.
Aspect 3: The method of aspect 2, wherein determining the portion of the bone as depicted in the ultrasound image that corresponds to the portion of the bone as depicted in the reference data comprises: generating curve data that characterizes a curve of the bone as depicted in the ultrasound image; and searching the bone as depicted in the reference data for a curve that matches the curve of the bone as depicted in the ultrasound image.
Aspect 4: The method of any of aspects 1-3, wherein determining the physical location of the ultrasound probe comprises determining the physical location of the ultrasound probe based on data from one or more sensors of the MR visualization device.
Aspect 5: The method of any of aspects 1-4, wherein the reference data comprises a plurality of computed tomography (CT) images of the bone.
Aspect 6: The method of any of aspects 1-5, wherein the reference data comprises a 3-dimensional model of the bone.
Aspect 7: The method of any of aspects 1-6, wherein generating the virtual guidance comprises generating a virtual directional element that indicates a direction the clinician is to move the ultrasound probe.
Aspect 8: The method of any of aspects 1-7, wherein causing the MR visualization device to output the virtual guidance to the clinician comprises: causing the MR visualization device to output the virtual guidance so that the virtual guidance appears to the clinician to be superimposed on the patient.
Aspect 9: The method of any of aspects 1-8, wherein: the registration data is first registration data, and generating the virtual guidance comprises: obtaining an estimated model of the soft tissue structure based on the reference data; generating, based on the first registration data, second registration data that registers virtual locations on the estimated model of the soft tissue structure and corresponding physical locations on the bone; and determining, based on the second registration data and the physical location of the ultrasound probe, a direction to move the ultrasound probe so that the ultrasound probe is at the target position.
Aspect 10: The method of aspect 9, wherein obtaining the estimated model of the soft tissue structure comprises generating the estimated model of the soft tissue structure as a statistical shape model of the soft tissue structure based on the reference data
Aspect 11: The method of any of aspects 1-10, wherein the soft tissue structure is one of: a tendon, a ligament, a muscle, cartilage, or a blood vessel.
Aspect 12: The method of any of aspects 1-11, wherein: the target position is a first target position, the virtual guidance is first virtual guidance, and the method further comprises: obtaining the second ultrasound data; determining second virtual guidance based on the reference data, the registration data, and the physical location of the ultrasound probe, wherein the second virtual guidance instructs the clinician how to move the ultrasound probe to that the ultrasound probe is in a second target position to generate third ultrasound data that provides additional information regarding the soft tissue structure of the patient; and causing the MR visualization device to output the second virtual guidance to the clinician.
Aspect 13: The method of any of aspects 1-12, wherein the method further comprises causing the MR visualization device to output a model of the bone and a model of the soft tissue structure so that the model of the bone and the model of the soft tissue structure appear to the clinician to be superimposed on the patient.
Aspect 14: The method of aspect 13, wherein the virtual guidance indicates how to adjust an angle of the ultrasound probe relative to the patient so that the ultrasound probe is in the target position to generate the second ultrasound data.
Aspect 15: A system includes a memory configured to store reference data depicting a bone of a patient; processing circuitry configured to: determine a physical location of an ultrasound probe; generate, based on first ultrasound data generated by the ultrasound probe, registration data that registers virtual locations on the bone of the patient as depicted in the reference data with corresponding physical locations on the bone of the patient; generate virtual guidance based on the reference data, the registration data, and the physical location of the ultrasound probe, wherein the virtual guidance instructs a clinician how to move the ultrasound probe so that the ultrasound probe is at a target position to generate second ultrasound data that provides information regarding a soft tissue structure of the patient; and cause a head-mounted Mixed Reality (MR) visualization device to output the virtual guidance to the clinician.
Aspect 16: The system of aspect 15, wherein: the physical location of the ultrasound probe is a physical location at which the ultrasound probe generated the first ultrasound data, and the processing circuitry is configured to, as part of generating the registration data: obtain an ultrasound image based on the first ultrasound data; determine a portion of the bone as depicted in the ultrasound image that corresponds to a portion of the bone as depicted in the reference data; generate displacement data describing a spatial displacement between the ultrasound probe and the portion of the bone depicted in the ultrasound image; and generate the registration data based on the physical location of the ultrasound probe and the displacement data.
Aspect 17: The system of aspect 16, wherein the processing circuitry is configured to, as part of determining the portion of the bone as depicted in the ultrasound image that corresponds to the portion of the bone as depicted in the reference data: generate curve data that characterizes a curve of the bone as depicted in the ultrasound image; and search the bone as depicted in the reference data for a curve that matches the curve of the bone as depicted in the ultrasound image.
Aspect 18: The system of any of aspects 15-17, wherein the processing circuitry is configured to, as part of determining the physical location of the ultrasound probe, determine the physical location of the ultrasound probe based on data from one or more sensors of the MR visualization device.
Aspect 19: The system of any of aspects 15-18, wherein the reference data comprises a plurality of computed tomography (CT) images of the bone.
Aspect 20: The system of any of aspects 15-19, wherein the reference data comprises a 3-dimensional model of the bone.
Aspect 21: The system of any of aspects 15-20, wherein the processing circuitry is configured to, as part of generating the virtual guidance, generate a virtual directional element that indicates a direction the clinician is to move the ultrasound probe.
Aspect 22: The system of aspect 21, wherein the processing circuitry is configured to, as part of causing the MR visualization device to output the virtual guidance to the clinician: cause the MR visualization device to output the virtual guidance so that the virtual guidance appears to the clinician to be superimposed on the patient.
Aspect 23: The system of any of aspects 15-22, wherein: the registration data is first registration data, and the processing circuitry is configured to, as part of generating the virtual guidance: obtain an estimated model of the soft tissue structure based on the reference data; generate, based on the first registration data, second registration data that registers virtual locations on the estimated model of the soft tissue structure and corresponding physical locations on the bone; and determine, based on the second registration data and the physical location of the ultrasound probe, a direction to move the ultrasound probe so that the ultrasound probe is at the target position.
Aspect 24: The system of aspect 23, wherein the processing circuitry is configured to, as part of obtaining the estimated model of the soft tissue structure, generate the estimated model of the soft tissue structure as a statistical shape model of the soft tissue structure based on the reference data.
Aspect 25: The system of any of aspects 15-24, wherein the soft tissue structure is one of: a tendon, a ligament, a muscle, cartilage, or a blood vessel.
Aspect 26: The system of any of aspects 15-26, wherein: the target position is a first target position, the virtual guidance is first virtual guidance, and the processing circuitry is further configured to: obtain the second ultrasound data; determine second virtual guidance based on the reference data, the registration data, and the physical location of the ultrasound probe, wherein the second virtual guidance instructs the clinician how to move the ultrasound probe to that the ultrasound probe is in a second target position to generate third ultrasound data that provides additional information regarding the soft tissue structure of the patient; and cause the MR visualization device to output the second virtual guidance to the clinician.
Aspect 27: The system of any of aspects 15-26, wherein the processing circuitry is further configured to cause the MR visualization device to output a model of the bone and a model of the soft tissue structure so that the model of the bone and the model of the soft tissue structure appear to the clinician to be superimposed on the patient.
Aspect 28: The system of aspect 27, wherein the virtual guidance indicates how to adjust an angle of the ultrasound probe relative to the patient so that the ultrasound probe is in the target position to generate the second ultrasound data.
Aspect 29: A computer-readable medium having instructions stored thereon that, when executed, cause processing circuitry to performing the methods of any of aspects 1-14.
Aspect 30: A system comprising means for performing the methods of any of aspects 1-14.

While the techniques been disclosed with respect to a limited number of examples, those skilled in the art, having the benefit of this disclosure, will appreciate numerous modifications and variations there from. For instance, it is contemplated that any reasonable combination of the described examples may be performed. It is intended that the appended claims cover such modifications and variations as fall within the true spirit and scope of the invention.

It is to be recognized that depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

In one or more examples, the functions described may be implemented in hardware, software, firmware, or any combination thereof. If implemented in software, the functions may be stored on or transmitted over as one or more instructions or code on a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

By way of example, and not limitation, such computer-readable storage media can comprise RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transitory media, but are instead directed to non-transitory, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Operations described in this disclosure may be performed by one or more processors, which may be implemented as fixed-function processing circuits, programmable circuits, or combinations thereof, such as one or more digital signal processors (DSPs), general purpose microprocessors, application specific integrated circuits (ASICs), field programmable gate arrays (FPGAs), or other equivalent integrated or discrete logic circuitry. Fixed-function circuits refer to circuits that provide particular functionality and are preset on the operations that can be performed. Programmable circuits refer to circuits that can programmed to perform various tasks and provide flexible functionality in the operations that can be performed. For instance, programmable circuits may execute instructions specified by software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. Accordingly, the terms “processor” and “processing circuity,” as used herein may refer to any of the foregoing structures or any other structure suitable for implementation of the techniques described herein.

1. A method comprising:
obtaining reference data depicting a bone of a patient;
determining a physical location of an ultrasound probe;
generating, based on first ultrasound data generated by the ultrasound probe, registration data that registers virtual locations on the bone of the patient as depicted in the reference data with corresponding physical locations on the bone of the patient;
generating virtual guidance based on the reference data, the registration data, and the physical location of the ultrasound probe, wherein the virtual guidance provides guidance to a clinician regarding how the ultrasound probe is positioned relative to a target position at which the ultrasound probe is able to generate second ultrasound data that provides information regarding a soft tissue structure of the patient; and
causing a head-mounted Mixed Reality (MR) visualization device to output the virtual guidance to the clinician.

2. The method of claim 1, wherein:
the physical location of the ultrasound probe is a physical location at which the ultrasound probe generated the first ultrasound data, and generating the registration data comprises:
obtaining an ultrasound image based on the first ultrasound data;
determining a portion of the bone as depicted in the ultrasound image that corresponds to a portion of the bone as depicted in the reference data;
generating displacement data describing a spatial displacement between the ultrasound probe and the portion of the bone depicted in the ultrasound image; and
generating the registration data based on the physical location of the ultrasound probe and the displacement data.

3. The method of claim 2, wherein determining the portion of the bone as depicted in the ultrasound image that corresponds to the portion of the bone as depicted in the reference data comprises:
generating curve data that characterizes a curve of the bone as depicted in the ultrasound image; and
searching the bone as depicted in the reference data for a curve that matches the curve of the bone as depicted in the ultrasound image.

4. The method of claim 1, wherein determining the physical location of the ultrasound probe comprises determining the physical location of the ultrasound probe based on data from one or more sensors of the MR visualization device.

5. The method of claim 1, wherein the reference data comprises a plurality of computed tomography (CT) images of the bone.

6. The method of claim 1, wherein the reference data comprises a 3-dimensional model of the bone.

7. The method of claim 1, wherein generating the virtual guidance comprises generating a virtual directional element that indicates a direction the clinician is to move the ultrasound probe.

8. The method of claim 1, wherein causing the MR visualization device to output the virtual guidance to the clinician comprises: causing the MR visualization device to output the virtual guidance so that the virtual guidance appears to the clinician to be superimposed on the patient.

9. The method of claim 1, wherein:
the registration data is first registration data, and generating the virtual guidance comprises:
obtaining an estimated model of the soft tissue structure based on the reference data;
generating, based on the first registration data, second registration data that registers virtual locations on the estimated model of the soft tissue structure and corresponding physical locations on the bone; and
determining, based on the second registration data and the physical location of the ultrasound probe, a direction to move the ultrasound probe so that the ultrasound probe is at the target position.

10. The method of claim 9, wherein obtaining the estimated model of the soft tissue structure comprises generating the estimated model of the soft tissue structure as a statistical shape model of the soft tissue structure based on the reference data.

11. The method of claim 1, wherein the soft tissue structure is one of: a tendon, a ligament, a muscle, cartilage, or a blood vessel.

12. The method of claim 1, wherein:
the target position is a first target position,
the virtual guidance is first virtual guidance, and the method further comprises:
obtaining the second ultrasound data;
determining second virtual guidance based on the reference data, the registration data, and the physical location of the ultrasound probe, wherein the second virtual guidance instructs the clinician how to move the ultrasound probe to that the ultrasound probe is in a second target position to generate third ultrasound data that provides additional information regarding the soft tissue structure of the patient; and
causing the MR visualization device to output the second virtual guidance to the clinician.

13. The method of claim 1, wherein the method further comprises causing the MR visualization device to output a model of the bone and a model of the soft tissue structure so that the model of the bone and the model of the soft tissue structure appear to the clinician to be superimposed on the patient.

14. The method of claim 13, wherein the virtual guidance indicates how to adjust an angle of the ultrasound probe relative to the patient so that the ultrasound probe is in the target position to generate the second ultrasound data.

15. A system comprising:
a memory configured to store reference data depicting a bone of a patient;
processing circuitry configured to:
determine a physical location of an ultrasound probe;
generate, based on first ultrasound data generated by the ultrasound probe, registration data that registers virtual locations on the bone of the patient as depicted in the reference data with corresponding physical locations on the bone of the patient;
generate virtual guidance based on the reference data, the registration data, and the physical location of the ultrasound probe, wherein the virtual guidance instructs a clinician how to move the ultrasound probe so that the ultrasound probe is at a target position to generate second ultrasound data that provides information regarding a soft tissue structure of the patient; and
cause a head-mounted Mixed Reality (MR) visualization device to output the virtual guidance to the clinician.

16. The system of claim 15, wherein:
the physical location of the ultrasound probe is a physical location at which the ultrasound probe generated the first ultrasound data, and
the processing circuitry is configured to, as part of generating the registration data:
obtain an ultrasound image based on the first ultrasound data;
determine a portion of the bone as depicted in the ultrasound image that corresponds to a portion of the bone as depicted in the reference data;
generate displacement data describing a spatial displacement between the ultrasound probe and the portion of the bone depicted in the ultrasound image; and
generate the registration data based on the physical location of the ultrasound probe and the displacement data.

17. The system of claim 16, wherein the processing circuitry is configured to, as part of determining the portion of the bone as depicted in the ultrasound image that corresponds to the portion of the bone as depicted in the reference data:
generate curve data that characterizes a curve of the bone as depicted in the ultrasound image; and
search the bone as depicted in the reference data for a curve that matches the curve of the bone as depicted in the ultrasound image.

18. The system of claim 15, wherein the processing circuitry is configured to, as part of determining the physical location of the ultrasound probe, determine the physical location of the ultrasound probe based on data from one or more sensors of the MR visualization device.

19. The system of claim 15, wherein the reference data comprises a plurality of computed tomography (CT) images of the bone.

20. The system of claim 15, wherein the reference data comprises a 3-dimensional model of the bone.

21. The system of claim 15, wherein the processing circuitry is configured to, as part of generating the virtual guidance, generate a virtual directional element that indicates a direction the clinician is to move the ultrasound probe.

22. The system of claim 21, wherein the processing circuitry is configured to, as part of causing the MR visualization device to output the virtual guidance to the clinician: cause the MR visualization device to output the virtual guidance so that the virtual guidance appears to the clinician to be superimposed on the patient.

23. The system of claim 15, wherein:
the registration data is first registration data, and
the processing circuitry is configured to, as part of generating the virtual guidance:
obtain an estimated model of the soft tissue structure based on the reference data;
generate, based on the first registration data, second registration data that registers virtual locations on the estimated model of the soft tissue structure and corresponding physical locations on the bone; and
determine, based on the second registration data and the physical location of the ultrasound probe, a direction to move the ultrasound probe so that the ultrasound probe is at the target position.

24. The system of claim 23, wherein the processing circuitry is configured to, as part of obtaining the estimated model of the soft tissue structure, generate the estimated model of the soft tissue structure as a statistical shape model of the soft tissue structure based on the reference data.

25. The system of claim 15, wherein the soft tissue structure is one of: a tendon, a ligament, a muscle, cartilage, or a blood vessel.

26. The system of claim 15, wherein:
the target position is a first target position,
the virtual guidance is first virtual guidance, and the processing circuitry is further configured to:
obtain the second ultrasound data;
determine second virtual guidance based on the reference data, the registration data, and the physical location of the ultrasound probe, wherein the second virtual guidance instructs the clinician how to move the ultrasound probe to that the ultrasound probe is in a second target position to generate third ultrasound data that provides additional information regarding the soft tissue structure of the patient; and
cause the MR visualization device to output the second virtual guidance to the clinician.

27. The system of claim 15, wherein the processing circuitry is further configured to cause the MR visualization device to output a model of the bone and a model of the soft tissue structure so that the model of the bone and the model of the soft tissue structure appear to the clinician to be superimposed on the patient.

28. The system of claim 27, wherein the virtual guidance indicates how to adjust an angle of the ultrasound probe relative to the patient so that the ultrasound probe is in the target position to generate the second ultrasound data.

29. One or more non-transitory computer-readable storage media comprising instructions stored thereon that, when executed by processing circuitry, cause the processing circuitry to:
obtaining reference data depicting a bone of a patient;
determining a physical location of an ultrasound probe;
generating, based on first ultrasound data generated by the ultrasound probe, registration data that registers virtual locations on the bone of the patient as depicted in the reference data with corresponding physical locations on the bone of the patient;
generating virtual guidance based on the reference data, the registration data, and the physical location of the ultrasound probe, wherein the virtual guidance provides guidance to a clinician regarding how the ultrasound probe is positioned relative to a target position at which the ultrasound probe is able to generate second ultrasound data that provides information regarding a soft tissue structure of the patient; and
causing a head-mounted Mixed Reality (MR) visualization device to output the virtual guidance to the clinician.

30. (canceled)
